(19) United States
(12) Patent Application Publication
(10) Pub. No.: US 2018/0358116 A1
(43) Pub. Date: Dec. 13, 2018

(54) DYNAMIC DATA EXCHANGE PLATFORM FOR EMERGENCY MEDICAL SERVICES

(71) Applicant: Health EWay, Inc., Sierra Madre, CA (US)

(72) Inventors: Matthew Kristin, Lexington, MA (US); Gary Mendoza, Sierra Madre, CA (US)

(21) Appl. No.: 16/003,004

(22) Filed: Jun. 7, 2018

Related U.S. Application Data
(60) Provisional application No. 62/516,624, filed on Jun. 7, 2017.

(51) Int. Cl. G16H 10/60; G16H 70/20; G08G 1/0968; G06Q 40/08

(52) U.S. Cl. CPC G16H 10/60; G06Q 40/08; G08G 1/096855; G16H 70/20

(57) ABSTRACT

A method for a cloud-based exchange of emergency medical service (EMS) data is provided. The method can include receiving, from a client, patient data associated with a patient. At least one payer for the patient can be identified by sending a first query to a first membership database at a first payer system and a second query to a second membership database at a second payer system. The first query and/or the second query can be generated based on the patient data. In response to a payer of the first payer system being identified as a payer for the patient, a third query can be sent to a provider database at the first payer system for retrieving a preferred in-network facility for the patient. A user interface can be generated for displaying, at the client, patient routing data including the preferred in-network facility.

### RELATED APPLICATION

This application claims priority to U.S. Provisional Application No. 62/516,624 entitled DYNAMIC DATA EXCHANGE PLATFORM FOR EMERGENCY MEDICAL SERVICES and filed on Jun. 7, 2017, the disclosure of which is incorporated herein by reference in its entirety.

### TECHNICAL FIELD

The subject matter described herein relates generally to cloud computing and more specifically to a cloud-based data exchange platform for emergency medical services (EMS).

### BACKGROUND

Patient data is increasingly being collected and stored in a digital format including, for example, electronic medical records (EMRs), electronic health records (EHRs), and/or the like. A multitude of entities can collect and store patient data including, for example, patients, healthcare professionals and paraprofessionals, hospitals, ambulatory clinics, health plans/insurance companies/payers, governmental agencies, third party service providers, research facilities, and/or the like. As one example, a typical electronic medical record (EMR) can contain a patient's health information including, for example, demographics, progress notes, problems, medications, vital signs, past medical history, immunizations, laboratory data, radiology reports, data from Bluetooth-enabled patient monitoring devices, clinical analytics, and/or the like. Alternatively and/or additionally, the data systems maintained by healthcare entities such as, for example, health plans, insurance companies, payers, and/or the like, can also contain the patient's benefit, financial, and payment information including, for example, delivery network configuration, benefit design, insurance coverage, claims information, risk stratification, business intelligence data, and/or the like.

To the extent that digitizing patient data can automate and streamline the workflow for medical diagnosis and treatment, digital patient data such as, for example, electronic medical records (EMRs), electronic health records (EHRs), and/or the like, can be vulnerable to loss, damage, theft, and/or other forms of misuse. As such, the use and disclosure of digital patient data can be subject to regulations including, for example, the privacy and security rules imposed by the Health Insurance Portability and Accountability Act (HIPAA) of 1996.

Ambulances have largely been unable to take advantage of the depth of available and relevant patient data. This can be attributed, in part, to the real-time, mobile nature of ambulance activities and the multiple data sources that could inform what happens in the ambulance. In addition, ambulances have historically been viewed primarily as transportation to the healthcare system instead of a critically important point in the continuum of care.

### SUMMARY

Systems, methods, and articles of manufacture, including computer program products, are provided for a cloud-based data exchange platform for emergency medical services (EMS). In some example embodiments, there is provided a system. The system may include at least one data processor and at least one memory. The at least one memory can store instructions that result operations when executed by the at least one data processor. The operations can include: receiving, from a client, patient data associated with a patient; identify at least one payer for the patient at least by sending a first query to a first membership database at a first payer system and a second query to a second membership database at a second payer system, the first query and/or the second query being generated based at least on the patient data; in response to a payer associated with the first payer system being identified as the at least one payer for the patient, sending, to a provider database at the first payer system, a third query for retrieving at least a first preferred in-network facility for the patient; and generating a user interface for displaying, at the client, patient routing data that includes the first preferred in-network facility.

In some variations, one or more features disclosed herein including the following features can optionally be included in any feasible combination. The patient routing data can include the first preferred in-network facility based at least on the first preferred in-network facility being compliant with a clinical protocol applicable to the patient. The clinical protocol applicable to the patient can specify a required procedure for treating, triaging, and/or transporting the patient. A protocol database storing a plurality of clinical protocols can be queried in order to identify the clinical protocol applicable to the patient. The patient routing data can be generated to include a second preferred in-network facility and/or an out-of-network facility instead of the first preferred in-network facility based at least on the first preferred in-network facility being non-compliant with the clinical protocol applicable to the patient.

In some variations, one or more features disclosed herein including the following features can optionally be included in any feasible combination. As an example, once the identity of the patient being transported is confirmed, the operations can include querying other data systems holding data relevant to that specific patient (e.g., data residing in EMRs, registries that report whether the patient has an advance care directive/POLST on file or is an registered organ donor) and providing that assembled patient-specific data to paramedics treating the patient.

In some variations, the patient routing data can include the first preferred in-network facility based at least on a diversion status of the first preferred in-network facility indicating whether the first preferred in-network facility is able to accept the patient. The diversion status for the first preferred in-network facility can be determined at least by querying a facility system and/or a third party system. The patient routing data can be generated to include a second preferred in-network facility and/or an out-of-network facility instead of the first preferred in-network facility based at least on the diversion status of the first preferred in-network facility indicating the first preferred in-network facility as being unable to accept the patient.

In some variations, the patient routing data can include the first preferred in-network facility based at least on the distance and/or traffic conditions to the first preferred in-network facility permitting the patient to be transported to the first preferred in-network facility within a threshold quantity of time. The distance and/or traffic conditions for the first preferred in-network facility can be determined by at least querying a navigation system. The patient routing data can be generated to include a second preferred in-network facility and/or an out-of-network facility instead of the first preferred in-network facility based at least on the distance and/or traffic conditions for the first preferred in-network facility preventing the patient to be transported to the first preferred in-network facility within a threshold quantity of time.

In some variations, the first query and/or the second query can be a Health Insurance Portability and Accountability Act (HIPAA) 270/271 transaction.

In some variations, at least one response can be received from the first membership database, the second membership database, and/or the provider database. Raw data included in the at least one response can be parsed by at least converting non-extensible markup language data into an extensible markup language format.

In some variations, the first membership database, the second membership database, and/or the provider database can each apply a different data format and/or data exchange protocol.

Non-transitory computer program products (i.e., physically embodied computer program products) are also described that store instructions, which when executed by one or more data processors of one or more computing systems, cause at least one data processor to perform operations herein. Similarly, computer systems are also described that may include one or more data processors and memory coupled to one or more data processors. The memory may temporarily or permanently store instructions that cause at least one processor to perform one or more of the operations described herein. In addition, methods can be implemented by one or more data processors either within a single computing system or distributed among two or more computing systems. Such computing systems can be connected and can exchange data and/or commands or other instructions or the like via one or more connections, including but not limited to a connection over a network (e.g., the Internet, a wireless wide area network, a local area network, a wide area network, a wired network, or the like), via a direct connection between one or more of the multiple computing systems, etc.

Implementations of the current subject matter can include, but are not limited to, methods consistent with the descriptions provided herein as well as articles that comprise a tangibly embodied machine-readable medium operable to cause one or more machines (e.g., computers, etc.) to result in operations implementing one or more of the described features. Similarly, computer systems are also described that may include one or more processors and one or more memories coupled to the one or more processors. A memory, which can include a non-transitory computer-readable or machine-readable storage medium, may include, encode, store, or the like one or more programs that cause one or more processors to perform one or more of the operations described herein. Computer implemented methods consistent with one or more implementations of the current subject matter can be implemented by one or more data processors residing in a single computing system or multiple computing systems. Such multiple computing systems can be connected and can exchange data and/or commands or other instructions or the like via one or more connections, including but not limited to a connection over a network (e.g. the Internet, a wireless wide area network, a local area network, a wide area network, a wired network, or the like), via a direct connection between one or more of the multiple computing systems, etc.

The details of one or more variations of the subject matter described herein are set forth in the accompanying drawings and the description below. Other features and advantages of the subject matter described herein will be apparent from the description and drawings, and from the claims. While certain features of the currently disclosed subject matter are described for illustrative purposes, it should be readily understood that such features are not intended to be limiting. The claims that follow this disclosure are intended to define the scope of the protected subject matter.

### DESCRIPTION OF DRAWINGS

The accompanying drawings, which are incorporated in and constitute a part of this specification, show certain aspects of the subject matter disclosed herein and, together with the description, help explain some of the principles associated with the disclosed implementations. In the drawings,

FIG. 1 depicts a system diagram illustrating an emergency medical service (EMS) data exchange system consistent with implementations of the current subject matter;

FIG. 2 depicts a block diagram illustrating a dynamic data exchange platform 110 consistent with implementations of the current subject matter;

FIG. 3 a flowchart illustrating a process for identifying one or more in-network facilities consistent with implementations of the current subject matter;

FIG. 4 depicts a flowchart illustrating a process for generating patient routing data consistent with implementations of the current subject matter;

FIG. 5 depicts a block diagram illustrating a computing system consistent with implementations of the current subject matter;

FIG. 6A depicts a user interface for a dynamic data exchange platform consistent with implementations of the current subject matter; and

FIG. 6B depicts a user interface for a dynamic data exchange platform consistent with implementations of the current subject matter.

When practical, similar reference numbers denote similar structures, features, or elements.

### DESCRIPTION

Many patients being transported in ambulances are sufficiently stable to be delivered to an appropriate in-network facility without compromising patient care or safety in any manner. However, emergency medical technicians (EMTs) are often unable to determine an appropriate in-network facility for a patient. As such, the patient is usually delivered to the nearest emergency room, even when that emergency room is part of an out-of-network facility and/or a less-preferred in-network facility. The consequences of being delivered to an out-of-network facility and/or a less-preferred in-network facility can include compromised patient care and excessive patient cost. For instance, an out-of-network facility and/or a less-preferred in-network facility may lack immediate access to the patient's medical records, which can delay patient care, subject the patient to unnecessary tests and treatment, and/or necessitate a subsequent transfer to a preferred in-network facility. Furthermore, the patient may incur higher co-payments if the patient is delivered to an out-of-network and/or a less-preferred in-network facility due to the benefit design and/or network configuration of the patient's health insurance coverage. Likewise, the payer providing insurance coverage to the patient can incur higher costs attributable to unnecessary or delayed care, the network status of different healthcare facilities, differences in the contracting relationships with healthcare facilities, and/or the like.

In some implementations of the current subject matter, a dynamic data exchange platform (DDEP) can provide, to an emergency medical service (EMS) client, patient routing data identifying at least one preferred in-network facility for a patient. The dynamic data exchange platform can generate the patient routing data based on raw data residing at one or more host systems such as, for example, payer systems, facility systems, navigation systems, and/or the like. Each host system can apply, to the raw data, a different data format and/or data exchange protocol. As such, the dynamic data exchange platform can support any data format and/or data exchange protocol such that the dynamic data exchange platform is capable of collecting raw data form a variety of host systems, regardless of the data format and/or data exchange protocol applied at each host system. The dynamic data exchange platform can be further configured to securitize, parse, and/or assemble the raw data to generate the patient routing data, which can subsequently be persisted at the dynamic data exchange platform and/or presented to the emergency medical service client.

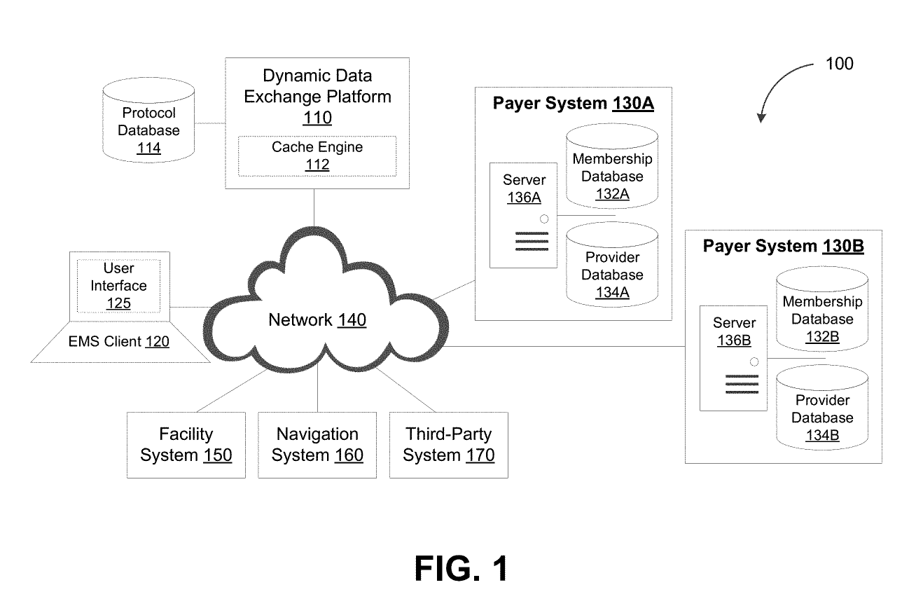

FIG. 1 depicts a system diagram illustrating an emergency medical service (EMS) data exchange system 100 consistent with implementations of the current subject matter. Referring to FIG. 1, the emergency medical service data exchange system 100 can include a dynamic data exchange platform 110.

As shown in FIG. 1, the dynamic data exchange platform 110 can be communicatively coupled, via a network 140, to a plurality of host systems including, for example, a first payer system 130A, a second payer system 130B, a facility system 150, a navigation system 160, a third party system 170, and/or the like. Alternatively and/or additionally, the dynamic data exchange platform 110 can also be communicatively coupled, via the network 140, to one or more emergency medical service (EMS) clients including, for example, an emergency medical service client 120. The network 140 can be any wired and/or wireless network including, for example, a public land mobile network (PLMN), a local area network (LAN), a wide area network (WLAN), a virtual local area network (VLAN), the Internet, and/or the like.

In some implementations of the current subject matter, the dynamic data exchange platform 110 can be configured to provide, to the emergency medical service client 120, patient routing data identifying at least one preferred in-network facility for a patient. The dynamic data exchange platform 110 can provide the patient routing data as part of an online service accessed via the network 140 including, for example, a cloud-based application, a web-based application, and/or the like. The emergency medical service client 120 can be deployed on an emergency medical service vehicle (e.g., an ambulance and/or the like) and can be any processor-based device capable of wired and/or wireless communication including, for example, a mobile telephone, a laptop computer, a desktop, a netbook, a tablet, a smartphone, a wearable apparatus (e.g., smartwatch), and/or the like. Alternatively and/or additionally, the emergency medical service client 120 can be an electronic patient care reporting (ePCR) system.

As shown in FIG. 1, the emergency medical service client 120 can interact with the dynamic data exchange platform 110 via a user interface 125 such as, for example, a graphic user interface (GUI) and/or the like. For example, at least some of the raw data required by the dynamic data exchange platform 110 to generate the patient routing data can be input at the emergency medical service client 120 via the user interface 125 before being sent to the dynamic data exchange platform 110 via the network 140. Alternatively and/or additionally, the patient routing data generated by the dynamic data exchange platform 110 can be sent, via the network 140, to the emergency medical service client 120 via the network 140 where the patient routing data can subsequently be outputted to the user interface 125.

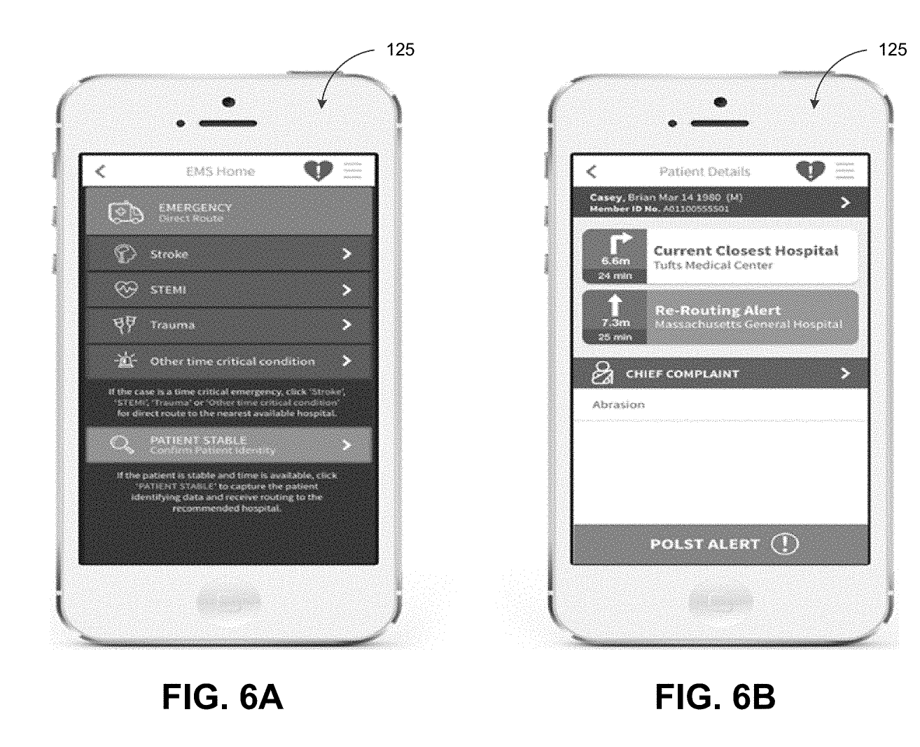

To further illustrate, FIGS. 6A-B depicts the user interface 125 for the dynamic data exchange platform 110 consistent with implementations of the current subject matter. For instance, the dynamic data exchange platform 110 can generate the user interface 125 shown in FIG. 6A for collecting, from the emergency medical service client 120, raw data. As shown in FIG. 6A, the user interface 125 can display a selection of patient conditions (e.g., stroke, STEMI, trauma, stable, and/or the like). Alternatively and/or additionally, the dynamic data exchange platform 110 can generate the user interface 125 shown in FIG. 6B for displaying, at the emergency medical service client 120, patient routing data. As shown in FIG. 6B, the patient routing data can include a facility that is currently closest to the patient as well as any rerouting alerts necessitated, for example, by the diversion status and/or traffic conditions associated with the closest facility.

As noted, the dynamic data exchange platform 110 can generate the patient routing data based on raw data input at the emergency medical service client 120. This raw data can include patient data including, for example, patient identifier, patient condition, patient location, and/or the like. Alternatively and/or additionally, the raw data required to generate the patient routing data can also reside at one or more host systems including, for example, the first payer system 130A, the second payer system 130B, the facility system 150, the navigation system 160, and/or the like. Accordingly, the dynamic data exchange platform 110 can query the one or more host systems to collect additional raw data required to generate the patient routing data. Furthermore, in some implementations of the current subject matter, the dynamic data exchange platform 110 can parse raw data collected from the one or more host systems before the raw data can be used to generate the patient routing data. Parsing the raw data can include converting non-extensible markup language (XML) raw data into an extensible markup language format. For instance, the dynamic data exchange platform 110 can also parse raw image data (e.g., a photograph of the patient's identification card, plan membership card, and/or the like) received from the emergency medical service client 120 and generate, based on the image data, patient data such as, for example, name, address, date of birth, gender, membership identifier, group number, plan type, coverage type, and/or the like. In addition and/or instead of being immediately used for generating the patient routing data, parsed raw data can also be persisted at the dynamic data exchange platform 110, for example, in a cache engine 112.

To further illustrate, the dynamic data exchange platform 110 can query a first membership database 132A at the first payer system 130A and/or a second membership database 132B at the second payer system 130B to verify, based on at least a portion of the patient data, health insurance coverage for the patient. For instance, the dynamic data exchange platform 110 can send, to the first membership database 132A at the first payer system 130A and/or the second membership database 132B at the second payer system 130B, one or more queries generated based at least on the patient data. It should be appreciated that the dynamic data exchange platform 110 can query any number of payer systems. Furthermore, the dynamic data exchange platform 110 can query multiple payer systems simultaneously, for example, by sending parallel queries to each payer system.

Upon verifying health insurance coverage for the patient, the dynamic data exchange platform 110 can further query a first provider database 132A at the first payer system 130A and/or a second provider database 132B at the second payer system 130B to identify at least one preferred in-network facility for the patient. It should be appreciated that the first provider database 132A and/or the second provider database 132B can store the network configuration and/or the benefit design associated with the respective payers of the first payer system 132A and/or the second payer system 132B. Alternatively and/or additionally, the dynamic data exchange platform 110 can query the third party system 170 in order to identify at least one preferred in-network facility for the patient. The dynamic data exchange platform 110 may query the third party system 170 instead of the first provider database 132A and/or the second provider database 132B if the network configuration and/or benefit design of the payer associated with the first payer system 132A and/or the second payer system 132B are maintained by an independent third party.

In some implementations of the current subject matter, the patient data received from the emergency medical service client 120 may not identify a payer for the patient. As such, the dynamic data exchange platform 110 can query multiple payer systems (e.g., the first payer system 130A and the second payer 130B) to identify at least one payer providing health insurance coverage for the patient. Alternatively and/or additionally, if the patient data received from the emergency medical service client 120 does identify at least one payer for the patient, the dynamic data exchange platform 110 can query the corresponding payer systems to verify the at least one payer that does provide health insurance coverage for the patient. Furthermore, the dynamic data exchange platform 110 can be configured to respond to the receipt of patient data from the emergency medical service client 120 by simultaneously querying multiple host systems including, for example, the first payer system 130A, the second payer system 130B, the facility system 150, the navigation system 160, and/or the like. That is, the dynamic data exchange platform 110 may send, at a same time and/or substantially at a same time, multiple separate queries the first payer system 130A, the second payer system 130B, the facility system 150, the navigation system 160, and/or the like. In doing so, the dynamic data exchange platform 110 may be capable of providing, to the emergency medical service client 120, the patient routing data in real time and/or in near real time.

In some implementations of the current subject matter, the dynamic data exchange platform 110 can determine, by querying the first membership database 132A of the first payer system 130A and the second membership database 132B of the second payer system 130B, that the patient receives health insurance coverage from the payer associated with the first payer system 130A and the payer associated with the second payer system 130B. When that is the case, the dynamic data exchange platform 110 may further query the first payer system 130A (e.g., the first membership database 132A) and/or the second payer system 130B (e.g., the second membership database 132B) to determine which payer provides primary coverage for the patient and which payer provides secondary coverage for the patient.

As used herein, a preferred in-network facility can refer to a facility (e.g., a hospital and/or the like) that accepts a health insurance coverage offered by a payer, for example, associated with the first payer system 130A and/or the second payer system 130B. It should be appreciated that a payer can be associated with multiple preferred in-network facilities and the preferences of these facilities can be tiered such that some in-network facilities may be more preferred than other in-network facilities. As used herein, a payer can refer to any entity other than a patient that finances, reimburses, and/or otherwise assumes financial responsible for the cost of healthcare services. For example, a payer can be a health plan, a health insurance company, a government agency, a government program, an employer sponsor of self-funded benefit programs, and/or the like.

The first membership database 132A, the second membership database 132B, the first provider database 134A, and/or the second provider database 134B can each apply a different data format and/or a data exchange protocol. Thus, in some implementations of the current subject matter, the dynamic data exchange platform 110 can support any data format and/or data exchange protocol, and can therefore be capable of collecting raw data form a variety of host systems, regardless of the data format and/or data exchange protocol applied at each host system. For example, the dynamic data exchange platform 110 may support various data formats including, for example, comma delimited files, databases, XML-based healthcare standard formats, non-standard XML formats, legacy formats, and/or the like. Moreover, the dynamic data exchange platform 110 can use different types of queries for querying the first membership database 132A, the second membership database 132B, the first provider database 134A, and/or the second provider database 134B. For instance, in order to verify that the patient has health insurance coverage with a payer associated with the first payer system 130A and/or the second payer system 130B, the dynamic data exchange platform 110 can use a Health Insurance Portability and Accountability Act (HIPAA) 270/271 transaction to query the first membership database 132A at the first payer system 130A and/or the second membership database 132B at the second payer system 130B.

In some implementations of the current subject matter, transmissions between the dynamic data exchange platform 110 and the first payer system 130A and/or the second payer system 130B can include medical information subject to regulations such as, for example, the Health Insurance Portability and Accountability Act (HIPAA) of 1996. As such, the dynamic data exchange platform 110 can impose one or more measures for safeguarding the privacy and security of the data exchanged between the dynamic data exchange platform 110 and the first payer system 130A and/or the second payer system 130B. For example, the first payer system 130A and the second payer system 130B can each be assigned a unique identifier, which can be used by the dynamic data exchange platform 110 for validating the first payer system 130A and the second payer system 130B. Alternatively and/or additionally, the dynamic data exchange platform 110 can apply, to the transmissions between the dynamic data exchange platform 110 and the first payer system 130A and/or the second payer system 130B, encryption, digital keys, unique identifiers, auditing functions, and/or the like.

In some implementations of the current subject matter, the patient routing data can further be generated based on clinical protocols. For example, the dynamic data exchange platform 110 can maintain a protocol database 114 for storing one or more clinical protocols. Furthermore, the dynamic data exchange platform 110 can query, based at least on the patient data (e.g., patient identifiers, patient condition, patient location, and/or the like) received from the emergency medical service client 120, the protocol database 114 to identify a clinical protocol that is applicable to the patient. For instance, a specific clinical protocol may be applicable to the patient given the patient's condition (e.g., cardiovascular, obstetrical, gynecological, respiratory, trauma, toxin, and/or the like), location, and/or the like. That clinical protocol may specify, for example, the required procedures for treating, triaging, and/or transporting the patient. Accordingly, the applicable clinical protocol can, in at least some instances, dictate the facility to which to transport the patient. For example, the dynamic data exchange platform 110 can exclude a preferred in-network facility that does not have the appropriate scope of service for treating the patient's condition. According to some implementations of the current subject matter, the dynamic data exchange platform 110 can update the user interface 125 to display, at the emergency medical service client 120, at least a portion of the applicable clinical protocol.

In some implementations of the current subject matter, upon identifying at least one preferred in-network facility, the dynamic data exchange platform 110 can query the facility system 150 of the preferred in-network facility to determine a diversion status of that preferred in-network facility. However, it should be appreciated that the facility system 150 can be maintained by an independent third party having no affiliations with the preferred in-network facility. If the preferred in-network facility is “on diversion,” the preferred in-network facility may be unable to accept the patient and may therefore require the dynamic data exchange platform 110 to identify a different in-network facility and/or an out-of-network facility for the patient. It should be appreciated that the dynamic data exchange platform 110 may support any data format and/or data protocol. As such, the dynamic data exchange platform 110 may be able to query the facility system 150 and obtain the diversion status of the preferred in-network facility, regardless of the data format and/or data protocol applied by the facility system 150.

Alternatively and/or additionally, the dynamic data exchange platform 110 can query the navigation system 160 to determine traffic conditions for the preferred in-network facility. For example, the navigation system 160 can be associated with a global positioning system (GPS) based navigation service. In some implementations of the current subject matter, upon determining traffic conditions that prevent the patient from being transported to the preferred in-network facility within a threshold quantity of time (e.g., as specified by the applicable clinical protocol), the dynamic data exchange platform 110 can identify a different in-network facility and/or an out-of-network facility for the patient. For instance, the traffic conditions can indicate road closures, hazards, accidents, and/or other delays that can prevent the patient from being delivered to the preferred in-network facility in a timely manner. As noted, the dynamic data exchange platform 110 may support any data format and/or data protocol. Thus, the dynamic data exchange platform 110 may be able to query the navigation system 160 and obtain the traffic conditions for the preferred in-network facility, regardless of the data format and/or data protocol applied by the navigation system 160.

In some implementations of the current subject matter, upon identifying at least one in-network facility and/or out-of-network facility for the patient, the dynamic data exchange platform 110 can update the user interface 125 to display, at the emergency medical service client 120, the corresponding patient routing data. Furthermore, the dynamic data exchange platform 110 can further track whether the patient is transported to the preferred in-network facility as recommended by the patient routing data, a different in-network facility, and/or an out-of-network facility. For instance, according to some implementations of the current subject matter, the dynamic data exchange platform 110 can receive, from the emergency medical service client 120, a confirmation of the patient having been transported to the preferred in-network facility included in the patient routing data, a different in-network facility, and/or an out-of-network facility.

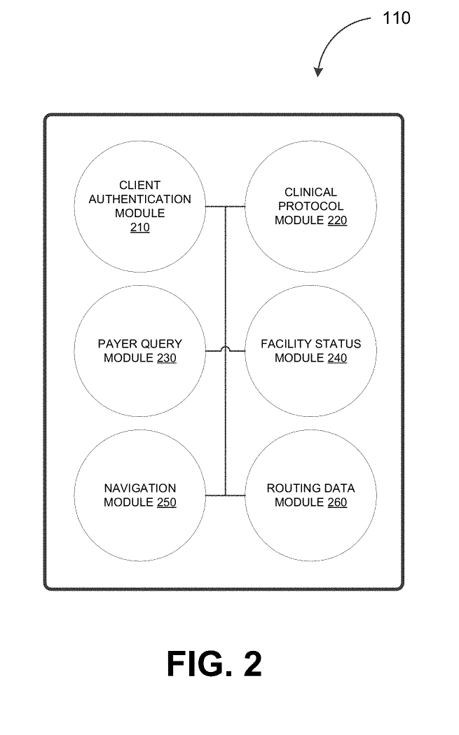

FIG. 2 depicts a block diagram illustrating the dynamic data exchange platform 110 consistent with implementations of the current subject matter. Referring to FIGS. 1-2, the dynamic data exchange platform 110 can include a client authentication module 210, a clinical protocol module 220, a payer query module 230, a facility status module 240, a navigation module 250, and a routing data module 260.

In some implementations of the current subject matter, the client authentication module 210 can be configured to authenticate the emergency medical service client 120. For instance, the emergency medical service client 120 can send, to the dynamic data exchange platform 110, authentication credentials including, for example, username, password, and/or the like. The client authentication module 210 can determine, based at least on the authentication credentials received from the emergency medical service client 120, whether the emergency medical service client 120 is authorized to access the dynamic data exchange platform 110 and/or the data available via the dynamic data exchange platform 110. It should be appreciated that access to the dynamic data exchange platform 110 and/or the data available from the dynamic data exchange platform 110 can be limited to clients that have been successfully authenticated by the client authentication module 210.

Upon the emergency medical service client 120 being successfully authenticated by the client authentication module 210, the dynamic data exchange platform 110 can receive, from the emergency medical service client 120, raw data that includes patient data. As noted, the patient data can include a patient identifier, a patient condition, a patient location, and/or the like. In some implementations of the current subject matter, the clinical protocol module 220 can be configured to identify, based at least on the patient data, a clinical protocol that is applicable to the patient. For example, the clinical protocol module 220 can query, based at least on the patient data, the protocol database 114 to identify a clinical protocol that is applicable to the patient given the patient's condition, location, and/or the like. The clinical protocol may specify, for example, the required procedures for treating, triaging, and/or transporting the patient. Thus, the applicable clinical protocol can, in at least some instances, dictate the facility to which to transport the patient and can therefore be part of the raw data used to generate the patient routing data.

In some implementations of the current subject matter, the payer query module 230 can be configured with advance logic features that consider multiple data elements to identify the preferred in-network facility, including availability of the patient's EMR records, patient expressed wishes, center of excellence designation, contractual pricing arrangements, network tiering arrangements, accountable care organization (ACO) participation, delivery system integration, shared risk arrangements and/or other such factors.

For instance, the payer query module 230 can be configured to query one or more payer systems including, for example, the first payer system 130A, the second payer system 130B, and/or the like. The payer query module 230 can query the first membership database 132A at the first payer system 130A and/or the second membership database 134B at the second payer system 130B in order to verify health insurance coverage for the patient. In doing so, the payer query module 230 may be able to identify at least one payer providing health insurance coverage for the patient, even when the patient data from the emergency medical service client 120 fails to identify a single payer for the patient. Upon verifying health insurance coverage for the patient, the dynamic data exchange platform 110 can further query the first provider database 132A at the first payer system 130A and/or the second provider database 132B at the second payer system 130B to identify at least one preferred in-network facility for the patient.

In some implementations of the current subject matter, the facility status module 240 can be configured to query the facility system 150 of the preferred in-network facility identified by the payer query module 230 to determine diversion status of the preferred in-network facility. As noted, the diversion status of the preferred in-network facility can indicate whether the preferred in-network facility is able to accept the patient. When the diversion status of the preferred in-network facility indicates that the preferred in-network facility is unable to accept the patient, the dynamic data exchange platform 110, for example, the payer query module 230, can be required to identify another in-network facility and/or an out-of-network facility for the patient.

In some implementations of the current subject matter, the navigation module 250 can be configured to query the navigation system 160 to determine traffic conditions for the preferred in-network facility. If traffic conditions indicate that the patient cannot be transported to the preferred in-network facility within a threshold quantity of time (e.g., as specified by the applicable clinical protocol), the dynamic data exchange platform 110, for example, the payer query module 230, can identify another in-network facility and/or an out-of-network facility for the patient.

In some implementations of the current subject matter, the routing data module 260 can be configured to generate patient routing data identifying at least one preferred in-network facility for the patient. The routing data module 260 can generate the patient routing data based on the applicable clinical protocol identified by the clinical protocol module 220, the at least one preferred in-network facility identified by the payer query module 230, the diversion status of the preferred in-network facilities determined by the facility status module 240, and/or the traffic conditions for the preferred in-network facilities determined by the navigation module 250.

For example, the routing data module 260 can generate the patient routing data to include a facility if that facility is a preferred in-network facility that complies with the applicable clinical protocol. Furthermore, the routing data module 260 can generate the patient routing data to include the facility if the diversion status of the facility indicates that the facility is able to accept the patient and the traffic conditions for the facility indicates that the patient can be transported to the facility within a threshold quantity of time. Alternatively and/or additionally, the routing data module 260 can generate the patient routing data to include a different in-network facility and/or an out-of-network facility that complies with the applicable clinical protocol if the diversion status of the facility indicates that the facility is unable to accept the patient and/or the traffic conditions for the facility indicates that the patient cannot be transported to the facility within a threshold quantity of time.

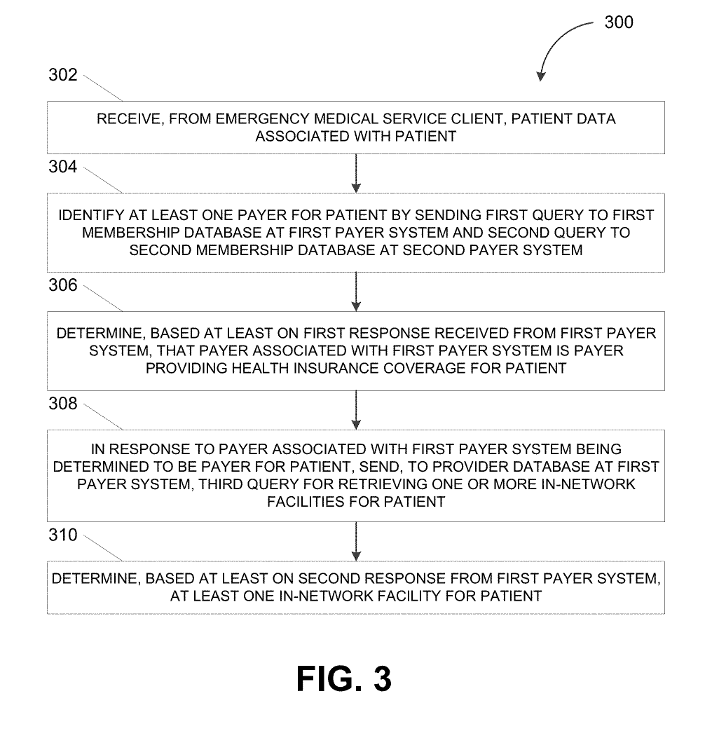

FIG. 3 a flowchart illustrating a process 300 for identifying one or more in-network facilities consistent with implementations of the current subject matter. Referring to FIGS. 1-3, the process 300 can be performed by the dynamic data exchange platform 110.

The dynamic data exchange platform 110 can receive, from the emergency medical service client 120, patient data associated with a patient (302). For example, the dynamic data exchange platform 110 can receive, from the emergency medical service client 120, patient data that includes patient identifiers, patient condition, patient location, and/or the like.

The dynamic data exchange platform 110 can identify at least one payer for the patient by sending a first query to a first membership database at a first payer system and a second query to a second membership database at a second payer system (304). In some implementations of the current subject matter, the dynamic data exchange platform 110, for example, the payer query module 230, can query the first membership database 132A at the first payer system 130A and/or the second membership database 132B. For instance, the payer query module 230 can send, the first membership database 132A at the first payer system 130A and/or the second membership database 132B, one or more queries generated based at least on the patient data. As noted, the dynamic data exchange platform 110 can query the first payer system 130A (e.g., the first membership database 132A) and the second payer system 130B (e.g., the second membership database 132B) simultaneously by at least sending the queries to the first payer system 130A and the second payer system 130B in parallel.

The dynamic data exchange platform 110 can query multiple payer systems if the patient data received from the emergency medical service client 120 does not identify a specific payer for the patient. Alternatively and/or additionally, the dynamic data exchange platform 110 can query a specific payer system if the patient data received from the emergency medical service client 120 identifies a specific payer for the patient.

The dynamic data exchange platform 110 can determine, based at least on a first response received from the first payer system, that a payer associated with the first payer system is a payer providing health insurance coverage for the patient (306). For example, the payer query module 230 at the dynamic data exchange platform 110 can receive, from the first payer system 130A and/or the second payer system 130B, a response verifying that a payer associated with the first payer system 130A and/or the second payer system 130B is providing health insurance coverage for the patient.

In response to the payer associated with the first payer system being determined to be the payer for the patient, the dynamic data exchange platform 110 can send, to a provider database at the first payer system, a third query for retrieving one or more in-network facilities for the patient (308). The dynamic data exchange platform 110 can determine, based at least on a second response from the first payer system, at least one preferred in-network facility for the patient (310). In some implementations of the current subject matter, the dynamic data exchange platform 110, for example, the payer query module 230, can further query the first provider database 134A at the first payer system 130A and/or the second provider database 134B to identify at least one preferred in-network facility for the patient.

As noted, the dynamic data exchange platform 110 can support any data format and/or data exchange protocol. Accordingly, the dynamic data exchange platform 110 may be capable of querying the first payer system 130A and/or the second payer system 130B, regardless of the data format and/or data exchange protocol applied at the first membership database 132A, the second membership database 132B, the first provider database 134A, and/or the second provider database 134B. For instance, in order to verify that the patient has health insurance coverage with a payer associated with the first payer system 130A and/or the second payer system 130B, the dynamic data exchange platform 110 can use a Health Insurance Portability and Accountability Act (HIPAA) 270/271 transaction to query the first membership database 132A at the first payer system 130A and/or the second membership database 132B at the second payer system 130B.

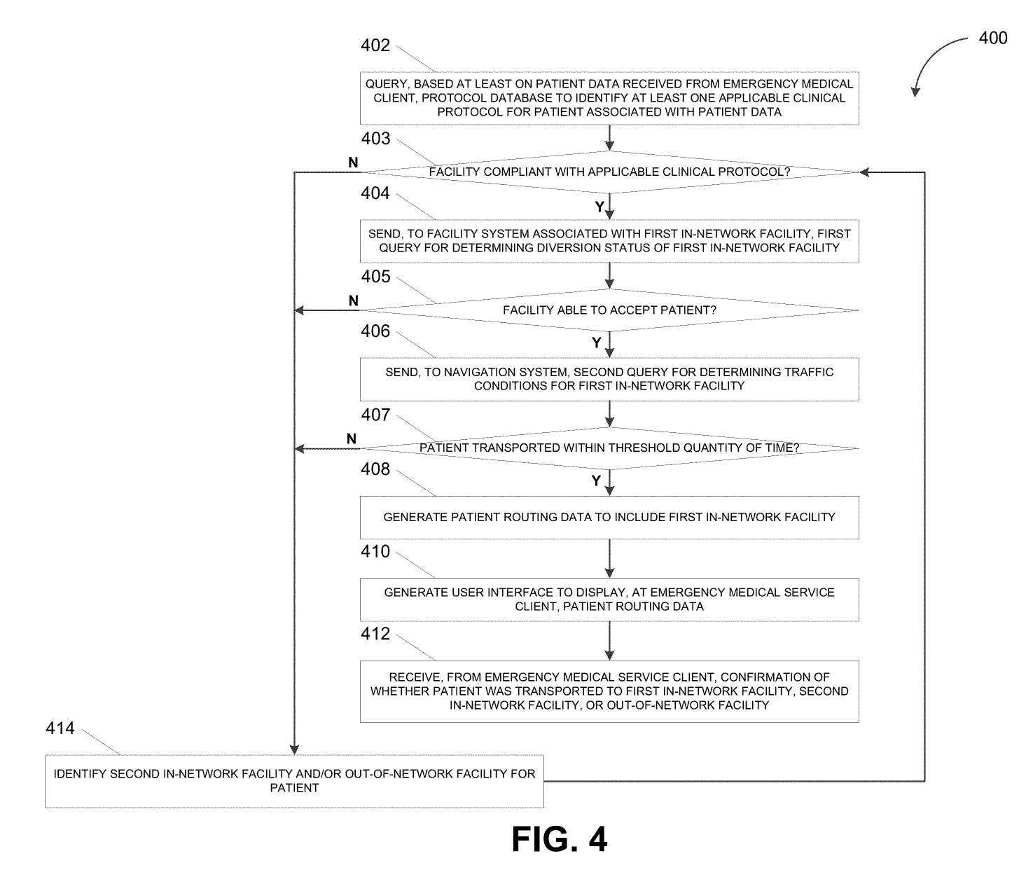

FIG. 4 depicts a flowchart illustrating a process for generating patient routing data consistent with implementations of the current subject matter. Referring to FIGS. 1-2 and 4, the process 400 can be performed by the dynamic data exchange platform 110.

The dynamic data exchange platform 110 can query, based at least on patient data received from the emergency medical service client 120, the protocol database 114 to identify at least one applicable clinical protocol for a patient associated with the patient data (402). In some implementations of the current subject matter, the dynamic data exchange platform 110 can maintain the protocol database 114 for storing one or more clinical protocols specifying, for example, the required procedures for treating, triaging, and/or transporting the patient. A different clinical protocol may be applicable to a patient depending on the patient's condition (e.g., cardiovascular, obstetrical, gynecological, respiratory, trauma, toxin, and/or the like), location, and/or the like). Accordingly, the dynamic data exchange platform 110, for example, the clinical protocol module 220, can query the protocol database 114 to identify at least one clinical protocol that is applicable to the patient based on patient data received from the emergency medical service client 120.

The dynamic data exchange platform 110 can determine whether a first preferred in-network facility for the patient is compliant with the applicable clinical protocol (403). As noted, the clinical protocol applicable to the patient can specify the required procedures for treating, triaging, and/or transporting the patient. For example, the scope of service offered by a preferred in-network facility can determine whether that in-network facility is compliant with the applicable clinical protocol. Accordingly, the applicable clinical protocol can, in at least some instances, dictate the facility to which the transport the patient. For example, some facilities may not comply with the applicable clinical protocol despite being a preferred in-network facility for the patient. Thus, the dynamic data exchange platform 110 can exclude a preferred in-network facility based on the preferred in-network facility not complying with the applicable clinical protocol.

If the dynamic data exchange platform 110 determines that the first preferred in-network facility for the patient is compliant with the applicable clinical protocol (403-Y), the dynamic data exchange platform 110 can send, to a facility system associated with the first preferred in-network facility, a first query for determining a diversion status of the first preferred in-network facility (404). For example, upon determining that a preferred in-network facility for the patient is also compliant with the applicable clinical protocol, the facility status module 240 at the dynamic data exchange platform 110 can query the facility system 150 of the preferred in-network facility to determine the diversion status of the preferred in-network facility. As noted, the diversion status of the preferred in-network facility can indicate whether the preferred in-network facility is able to accept the patient. Accordingly, the dynamic data exchange platform 110 can further determine, based a first response received from the facility system associated with the first preferred in-network facility, whether the first preferred in-network facility is able to accept the patient (405).

If the dynamic data exchange platform 110 determines that the first preferred in-network facility is able to accept the patient (405-Y), the dynamic data exchange platform 110 can send, to a navigation system, a second query for determining traffic conditions for the first preferred in-network facility (406). For example, in some implementations of the current subject matter, the navigation module 250 of the dynamic data exchange platform 110 can query the navigation system 160 to determine distance and/or traffic conditions to the preferred in-network facility. Furthermore, the dynamic data exchange platform 110 can determine, based at least on a second response received from the navigation system, whether the traffic conditions permit the patient to be transported to the first preferred in-network facility within a threshold quantity of time (407).

If the dynamic data exchange platform 110 determines that the traffic conditions does permit the patient to be transported to the first preferred in-network facility within a threshold quantity of time (407-Y), the dynamic data exchange platform 110 can generate patient routing data to include the first preferred in-network facility (408). Furthermore, the dynamic data exchange platform 110 can send, to the emergency medical service client 120, the patient routing data (410). For instance, the traffic conditions for the preferred in-network facility can indicate a lack of road closures, hazards, accidents, and/or other delays that can prevent the patient from being delivered to the preferred in-network facility in a timely manner, for example, as required by the applicable clinical protocol. Accordingly, the dynamic data exchange platform 110, for example, the routing data module 260, can generate patient routing data to include the preferred in-network facility. Furthermore, the dynamic data exchange platform 110 can generate and/or update the user interface 125 to display, at the emergency medical service client 120, the patient routing data.

The dynamic data exchange platform 110 can further receive, from the emergency medical service client 120, a confirmation of whether the patient was transported to the first preferred in-network facility, a second preferred in-network facility, or an out-of-network facility (412). In some implementations of the current subject matter, the dynamic data exchange platform 110 can further track whether the patient is transported to the preferred in-network facility included in the patient routing data, a different in-network facility, and/or an out-of-network facility. For example, the dynamic data exchange platform 110 can receive, from the emergency medical service client 120, a confirmation of the location to which the patient was ultimately transported.

The dynamic data exchange platform 110 can determine that the first preferred in-network facility for the patient is not compliant with the applicable clinical protocol (403-N). Alternatively and/or additionally, the dynamic data exchange platform 110 can also determine that the first preferred in-network facility is unable to accept the patient (405-N). The dynamic data exchange platform 110 can also determine that the traffic conditions does not permit the patient to be transported to the first preferred in-network facility within the threshold quantity of time (407-N). Under any of those circumstances, the dynamic data exchange platform 110 can identify a second preferred in-network facility and/or an out-of-network facility for the patient (414). The process 400 can continue at operation 403, where the dynamic data exchange platform 110 can determine whether the second preferred in-network facility and/or the out-of-network facility is compliant with the applicable clinical protocol.

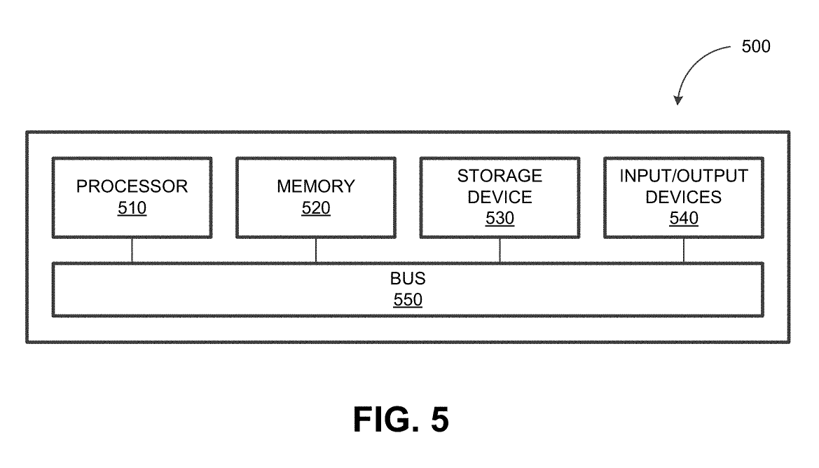

FIG. 5 depicts a block diagram illustrating a computing system 500, in accordance with some example embodiments. Referring to FIGS. 1 and 5, the computing system 500 can be used to implement the dynamic data exchange platform 110 and/or any components therein.

As shown in FIG. 5, the computing system 500 can include a processor 510, a memory 520, a storage device 530, and input/output devices 540. The processor 510, the memory 520, the storage device 530, and the input/output devices 540 can be interconnected via a system bus 550. The processor 510 is capable of processing instructions for execution within the computing system 500. Such executed instructions can implement one or more components of, for example, the dynamic data exchange platform 110. In some implementations of the current subject matter, the processor 510 can be a single-threaded processor. Alternately, the processor 510 can be a multi-threaded processor. The processor 510 is capable of processing instructions stored in the memory 520 and/or on the storage device 530 to display graphical information for a user interface provided via the input/output device 540.

The memory 520 is a computer readable medium such as volatile or non-volatile random-access memory (RAM) that stores information within the computing system 500. The memory 520 can store data structures representing configuration object databases, for example. The storage device 530 is capable of providing persistent storage for the computing system 500. The storage device 530 can be a floppy disk device, a hard disk device, an optical disk device, or a tape device, or other suitable persistent storage means. The input/output device 540 provides input/output operations for the computing system 500. In some implementations of the current subject matter, the input/output device 540 includes a keyboard and/or pointing device. In various implementations, the input/output device 540 includes a display unit for displaying graphical user interfaces.

According to some implementations of the current subject matter, the input/output device 540 can provide input/output operations for a network device. For example, the input/output device 540 can include Ethernet ports or other networking ports to communicate with one or more wired and/or wireless networks (e.g., a local area network (LAN), a wide area network (WAN), the Internet).

In some implementations of the current subject matter, the computing system 500 can be used to execute various interactive computer software applications that can be used for organization, analysis and/or storage of data in various (e.g., tabular) format (e.g., Microsoft Excel®, and/or any other type of software). Alternatively, the computing system 500 can be used to execute any type of software applications. These applications can be used to perform various functionalities, e.g., planning functionalities (e.g., generating, managing, editing of spreadsheet documents, word processing documents, and/or any other objects, etc.), computing functionalities, communications functionalities, etc. The applications can include various add-in functionalities or can be standalone computing products and/or functionalities. Upon activation within the applications, the functionalities can be used to generate the user interface provided via the input/output device 540. The user interface can be generated and presented to a user by the computing system 500 (e.g., on a computer screen monitor, etc.).

One or more aspects or features of the subject matter described herein may be realized in digital electronic circuitry, integrated circuitry, specially designed ASICs (application specific integrated circuits), computer hardware, firmware, software, and/or combinations thereof. These various implementations may include implementation in one or more computer programs that are executable and/or interpretable on a programmable system including at least one programmable processor, which may be special or general purpose, coupled to receive data and instructions from, and to transmit data and instructions to, a storage system, at least one input device (e.g., mouse, touch screen, etc.), and at least one output device.

These computer programs, which can also be referred to as programs, software, software applications, applications, components, or code, include machine instructions for a programmable processor, and can be implemented in a high-level procedural language, an object-oriented programming language, a functional programming language, a logical programming language, and/or in assembly/machine language. As used herein, the term “machine-readable medium” (sometimes referred to as a computer program product) refers to physically embodied apparatus and/or device, such as for example magnetic discs, optical disks, memory, and Programmable Logic Devices (PLDs), used to provide machine instructions and/or data to a programmable data processor, including a machine-readable medium that receives machine instructions as a machine-readable signal. The term “machine-readable signal” refers to any signal used to provide machine instructions and/or data to a programmable data processor. The machine-readable medium can store such machine instructions non-transitorily, such as for example as would a non-transient solid-state memory or a magnetic hard drive or any equivalent storage medium. The machine-readable medium can alternatively or additionally store such machine instructions in a transient manner, such as, for example, as would a processor cache or other random access memory associated with one or more physical processor cores.

To provide for interaction with a user, the subject matter described herein can be implemented on a computer having a display device, such as for example a cathode ray tube (CRT) or a liquid crystal display (LCD) monitor for displaying information to the user and a keyboard and a pointing device, such as for example a mouse or a trackball, by which the user may provide input to the computer. Other kinds of devices can be used to provide for interaction with a user as well. For example, feedback provided to the user can be any form of sensory feedback, such as for example visual feedback, auditory feedback, or tactile feedback; and input from the user may be received in any form, including, but not limited to, acoustic, speech, or tactile input. Other possible input devices include, but are not limited to, touch screens or other touch-sensitive devices such as single or multi-point resistive or capacitive trackpads, voice recognition hardware and software, optical scanners, optical pointers, digital image capture devices and associated interpretation software, and the like.

In some of the example described above, an administrator is described. The administrator may represent a fully and/or or partially automated computer-based process.

In the descriptions above and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it is used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” In addition, use of the term “based on,” above and in the claims is intended to mean, “based at least in part on,” such that an unrecited feature or element is also permissible.

The subject matter described herein may be implemented in a computing system that includes a back-end component (e.g., as a data server), or that includes a middleware component (e.g., an application server), or that includes a front-end component (e.g., a client computer having a graphical user interface or a Web browser through which a user may interact with an implementation of the subject matter described herein), or any combination of such back-end, middleware, or front-end components. The components of the system may be interconnected by any form or medium of digital data communication (e.g., a communication network). Examples of communication networks include a local area network (“LAN”), a wide area network (“WAN”), and the Internet.

The computing system may include clients and servers. A client and server are generally remote from each other and typically interact through a communication network. The relationship of client and server arises by virtue of computer programs running on the respective computers and having a client-server relationship to each other.

The subject matter described herein can be embodied in systems, apparatus, methods, and/or articles depending on the desired configuration. The implementations set forth in the foregoing description do not represent all implementations consistent with the subject matter described herein. Instead, they are merely some examples consistent with aspects related to the described subject matter. Although a few implementations have been described in detail above, other modifications or additions are possible. In particular, further features and/or implementations can be provided in addition to those set forth herein. For example, the implementations described above can be directed to various combinations and subcombinations of the disclosed features and/or combinations and subcombinations of several further features disclosed above. In addition, the logic flow(s) depicted in the accompanying figures and/or described herein do not necessarily require the particular order shown, or sequential order, to achieve desirable results. Other implementations may be within the scope of the following claim.

What is claimed is:

1. A system, comprising:
at least one data processor; and
at least one memory storing instructions, which when executed by at least one data processor, result in operations comprising:
receiving, from a client, patient data associated with a patient;
identify at least one payer for the patient by sending a first query to a first membership database at a first payer system and a second query to a second membership database at a second payer system, the first query and/or the second query being generated based at least on the patient data;
in response to a payer associated with the first payer system being identified as the at least one payer for the patient, sending, to a provider database at the first payer system, a third query for retrieving at least a first preferred in-network facility for the patient; and
generating a user interface for displaying, at the client, patient routing data that includes the first preferred in-network facility.

2. The system of claim 1, wherein the patient routing data includes the first preferred in-network facility based at least on the first preferred in-network facility being compliant with a clinical protocol applicable to the patient, and wherein the clinical protocol applicable to the patient specifies a required procedure for treating, triaging, and/or transporting the patient.

3. The system of claim 2, further comprising:
querying, based at least on the patient data, a protocol database to identify the clinical protocol applicable to the patient, the protocol database storing a plurality of clinical protocols; and
generating the patient routing data to include a second preferred in-network facility and/or an out-of-network facility instead of the first preferred in-network facility based at least on the first preferred in-network facility being non-compliant with the clinical protocol applicable to the patient.

4. The system of claim 1, wherein the patient routing data includes the first preferred in-network facility based at least on a diversion status of the first preferred in-network facility indicating the first preferred in-network facility as being able to accept the patient.

5. The system of claim 4, further comprising:
determining the diversion status for the first preferred in-network facility at least by querying a facility system; and
generating the patient routing data to include a second preferred in-network facility and/or an out-of-network facility instead of the first preferred in-network facility based at least on the diversion status of the first preferred in-network facility indicating the first preferred in-network facility as being unable to accept the patient.

6. The system of claim 1, wherein the patient routing data includes the first preferred in-network facility based at least on a traffic conditions for the first preferred in-network facility permitting the patient to be transported to the first preferred in-network facility within a threshold quantity of time.

7. The system of claim 6, further comprising:
determining the traffic conditions for the first preferred in-network facility by at least querying a navigation system; and
generating the patient routing data to include a second preferred in-network facility and/or an out-of-network facility instead of the first preferred in-network facility based at least on the traffic conditions for the first preferred in-network facility preventing the transport of the patient to the first preferred in-network facility within a threshold quantity of time.

8. The system of claim 1, wherein the first query and/or the second query comprise a Health Insurance Portability and Accountability Act (HIPAA) 270/271 transaction.

9. The system of claim 1, further comprising:
receiving, from the first membership database, the second membership database, and/or the provider database, at least one response; and
parsing raw data included in the at least one response by at least converting non-extensible markup language data into an extensible markup language format.

10. The system of claim 1, wherein the first membership database, the second membership database, and/or the provider database each applies a different data format and/or data exchange protocol.

11. A method, comprising:
receiving, from a client, patient data associated with a patient;
identifying at least one payer for the patient at least by sending a first query to a first membership database at a first payer system and a second query to a second membership database at a second payer system, the first query and/or the second query being generated based at least on the patient data;
in response to a payer associated with the first payer system being identified as the at least one payer for the patient, sending, to a provider database at the first payer system, a third query for retrieving at least a first preferred in-network facility for the patient; and
generating a user interface for displaying, at the client, patient routing data that includes the first preferred in-network facility.

12. The method of claim 11, wherein the patient routing data includes the first preferred in-network facility based at least on the first preferred in-network facility being compliant with a clinical protocol applicable to the patient, and wherein the clinical protocol applicable to the patient specifies a required procedure for treating, triaging, and/or transporting the patient.

13. The method of claim 12, further comprising:
querying, based at least on the patient data, a protocol database to identify the clinical protocol applicable to the patient, the protocol database storing a plurality of clinical protocols; and
generating the patient routing data to include a second preferred in-network facility and/or an out-of-network facility instead of the first preferred in-network facility based at least on the first preferred in-network facility being non-compliant with the clinical protocol applicable to the patient.

14. The method of claim 11, wherein the patient routing data includes the first preferred in-network facility based at least on a diversion status of the first preferred in-network facility indicating the first preferred in-network facility as being able to accept the patient.

15. The method of claim 14, further comprising:
determining the diversion status for the first preferred in-network facility at least by querying a facility system; and
generating the patient routing data to include a second preferred in-network facility and/or an out-of-network facility instead of the first preferred in-network facility based at least on the diversion status of the first preferred in-network facility indicating the first preferred in-network facility as being unable to accept the patient.

16. The method of claim 11, wherein the patient routing data includes the first preferred in-network facility based at least on a traffic conditions for the first preferred in-network facility permitting the patient to be transported to the first preferred in-network facility within a threshold quantity of time.

17. The method of claim 16, further comprising:
determining the traffic conditions for the first preferred in-network facility by at least querying a navigation system; and
generating the patient routing data to include a second preferred in-network facility and/or an out-of-network facility instead of the first preferred in-network facility based at least on the traffic conditions for the first preferred in-network facility preventing the patient to be transported to the first preferred in-network facility within a threshold quantity of time.

18. The method of claim 11, wherein the first query and/or the second query comprise a Health Insurance Portability and Accountability Act (HIPAA) 270/271 transaction.

19. The method of claim 1, further comprising:
receiving, from the first membership database, the second membership database, and/or the provider database, at least one response; and
parsing raw data included in the at least one response by at least converting non-extensible markup language data into an extensible markup language format.

20. A non-transitory computer-readable medium storing instructions, which when executed by at least one data processor, result in operations comprising:
receiving, from a client, patient data associated with a patient;
identifying at least one payer for the patient at least by sending a first query to a first membership database at a first payer system and a second query to a second membership database at a second payer system, the first query and/or the second query being generated based at least on the patient data;
in response to a payer associated with the first payer system being identified as the at least one payer for the patient, sending, to a provider database at the first payer system, a third query for retrieving at least a first preferred in-network facility for the patient; and
generating a user interface for displaying, at the client, patient routing data that includes the first preferred in-network facility.
